(19) United States
(12) Patent Application Publication
(10) Pub. No.: US 2002/0183597 A1
(43) Pub. Date: Dec. 5, 2002

(54) Microsensor for physiological pressure measurement

(76) Inventors: Kenton R. Kaufman, Rochester, MN (US); Thomas Wavering, Charlottesville, VA (US)

Correspondence Address: PAUL A. LEVY, Fish & Richardson P.C., Suite 2800, 45 Rockefeller Plaza, New York, NY, 10111, US

(21) Appl. No.: 10/132,894

(22) Filed: Apr. 25, 2002

Related U.S. Application Data
(60) Provisional application No. 60/287,095, filed on Apr. 25, 2001.

(51) Int. Cl. A61B 5/00

(52) U.S. Cl. 600/300; 600/546

(57) ABSTRACT

A body implantable sensor for sensing pressure in an environment within internal body tissue is described. In an implementation, the sensor includes a housing, an optical waveguide and a diaphragm with a reflective surface that faces a waveguide distal end and that reflects at least a portion of the signals exiting the waveguide distal end back into the waveguide. The diaphragm is movable relative to the waveguide end. The distance between the waveguide distal end and the diaphragm reflective surface is determined by pressure forces acting on a sensing surface of the diaphragm that is opposite the reflective surface. A cover surrounds the diaphragm and protects it from impingement, but leaves the diaphragm sensing surface exposed to pressure forces. In some implementations, an attachment mechanism stabilizes the sensor when implanted in the bodily tissue.

### CROSS-REFERENCE TO RELATED APPLICATION

This application claims priority from U.S. Provisional Application Ser. No. 60/287,095, filed Apr. 25, 2001 which is incorporated herein by reference in its entirety.

### STATEMENT AS TO FEDERALLY SPONSORED RESEARCH

This invention was made, at least in part, with funds from the Federal Government, awarded through the Department of Health and Human Services under contract HD3 1476. The government therefore has certain rights in the invention.

### TECHNICAL FIELD

This invention relates to a body implantable sensor for sensing pressure in an environment within internal body tissue.

### BACKGROUND OF THE INVENTION

Several devices and methods are currently used to measure biological pressure, such as intracranial pressure, blood vessel pressure, and intramuscular pressure. Two types of systems are available for recording pressure. One system is fluid filled and the other system uses a fiberoptic transducer. The fluid-filled systems include a needle manometer and the wick catheter technique for measurement of intramuscular pressure: However, pressure recording systems that are fluid filled may require infusion to maintain accuracy. Fluid-filled systems are sensitive to hydrostatic artifacts and may be used only with limited types of movement that do not involve limb position changes relative to the horizontal plane. In contrast, a fiberoptic transducer-tipped system is not sensitive to hydrostatic artifact and has been shown to be effective for measuring pressure.

Measurement of intramuscular pressure (IMP) is a measurement that relates to the timing and intensity of muscle contraction. Devices and methods that employ specialized apparata for these measurements may be referred to as “instrumented.” Instrumented devices and methods include hand-held dynamometers, hand grip dynamometers and isokinetic dynamometers. Electromyography has also been used to provide quantification of individual muscle function. An electromyogram (EMG) may be used to provide a quantitative measurement of muscle tension under isometric conditions. As opposed to electromyography, IMP can be used to quantify the contribution of both passive stretch of muscle and the active contraction of muscle.

### BRIEF SUMMARY OF THE INVENTION

In one aspect, a body implantable sensor for sensing pressure in an environment within internal body tissue is constructed from an optical waveguide, a diaphragm and a cover. The optical waveguide has a first end. The diaphragm has a reflective surface that faces the waveguide first end and that reflects signals exiting the waveguide back into the waveguide, the diaphragm being movable relative to the waveguide first end and the distance between the waveguide first end and the diaphragm reflective surface being determined by pressure forces acting on a sensing surface of the diaphragm that is opposite the reflective surface. A cover surrounding the diaphragm protects the diaphragm from impingement, but leaves the diaphragm sensing surface exposed to pressure forces.

“Impingement,” as used herein, includes, but is not limited to, physical forces, or mechanical forces acting upon the sensor while the sensor is being handled, being implanted, or while implanted in the body tissue.

This arrangement operates as a Fabry-Perot cavity. The diaphragm can move in response to physiological pressure, altering the distance between the diaphragm and the optical fiber. This alters the resultant optical signal, from which pressure may be inferred.

In another aspect, the invention is directed to a method of measuring physiological pressure including the steps of inserting the microsensor into an organism, anchoring the microsensor within the organism, transmitting light into the microsensor, receiving a returned light signal and processing the returned light signal to determine the physiological pressure.

In another aspect, the invention is directed to a method of measuring intramuscle pressure, including the steps of inserting a pressure microsensor into a muscle, anchoring the microsensor within the muscle, transmitting light into the microsensor, receiving a returned light signal, processing the returned light signal to determine intramuscular pressure, and processing the intramuscular pressure signal to infer muscle force.

Various implementations have the advantage of being immune to electromagnetic interference/radio frequency interference (EMI/RFI). Moreover, the sensor carries no electrical current.

Various implementations have a structure that enables production of a small size and light weight sensor. The cover in some implementations advantageously functions to protect the diaphragm from impingement.

It is a further advantage in some implementations that an attachment mechanism of the sensor stabilizes the sensor in the bodily tissue.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a system for optically sensing pressure inside a body.

FIG. 2 is a perspective diagram of an embodiment of an optical sensor that may be used in the system of FIG. 1.

FIG. 3 is a cross-sectional diagram of the sensor shown in FIG. 2.

FIG. 4 is a cross-sectional diagram of another embodiment of an optical sensor that may be used in the system of FIG. 1.

FIG. 5 is a cross-sectional diagram of yet another embodiment of an optical sensor that may be used in the system of FIG. 1.

FIG. 6 is a perspective diagram showing the sensor shown in FIG. 5 attached to body tissue.

FIG. 7 is a cross-sectional diagram of a further embodiment of an optical sensor that may be used in the system of FIG. 1.

FIG. 8 is a cross-sectional diagram of yet a further embodiment of an optical sensor that may be used in the system of FIG. 1.

FIG. 9 is a conceptual diagram illustrating the theory of operation of optical sensors of the type disclosed.

Like reference symbols in the various drawings indicate like elements.

### DETAILED DESCRIPTION OF THE INVENTION

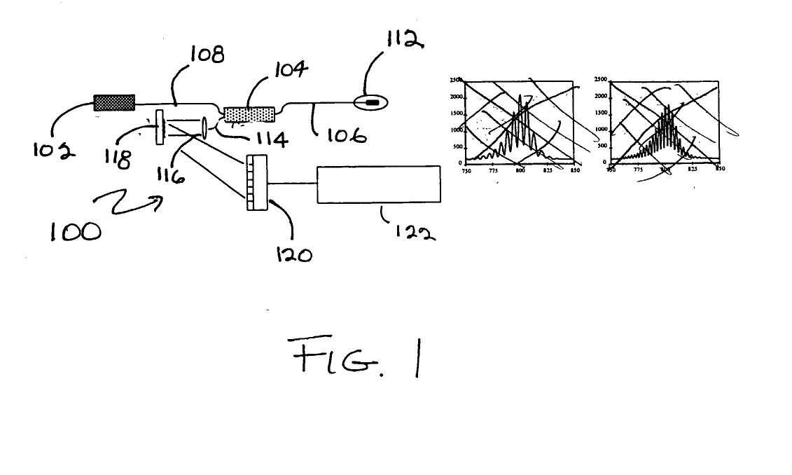

FIG. 1 is a block diagram of a low frequency, high resolution, fiber-optic measurement system 100. A light source 102 is used to supply an optical power signal through an optical waveguide 108. A coupler 104 couples the optical signal between waveguide 108 and a second waveguide 106 which conveys the optical signal to a sensor 112. Sensor 112 can provide optical return signals based upon sensor measurements. The sensor is made small enough to be implanted in bodily tissue and have the second waveguide couple the return signals to the external equipment. Return signals from the sensor are coupled to a waveguide 114 and focused by a lens 116 onto a diffraction grating 118. The diffraction grating serves to disperse the light into a series of spectra on both sides of the incident beam, and concentrate most of the energy into a single spectral order. The returned signal is then analyzed with a miniature spectrometer 120 coupled to a signal processor 122 that can determine a sensor measurement based upon a phase differential between returned signals.

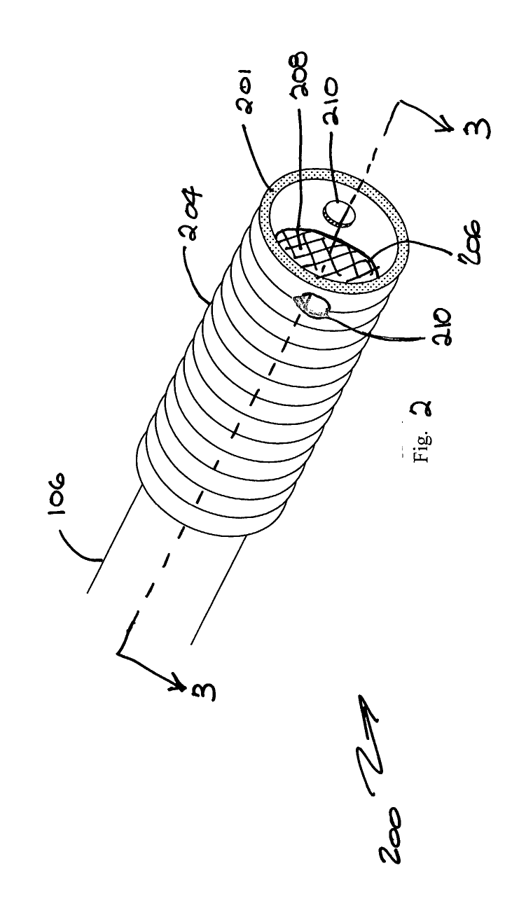

FIG. 2 is a perspective view of one implementation of a microsensor 200. The microsensor comprises a cover 201 attached to an end (not shown) of an optical waveguide 106. In this implementation, cover 201 has a threaded surface that stabilizes the sensor when it is implanted in bodily tissue such as muscle fibers. The threaded surface is one example of an attachment mechanism suitable for use with this implementation. Other attachment mechanisms may include tines, barbs, surface coatings or similar devices that engage the bodily tissue and stabilize the sensor when the sensor is implanted. A diaphragm 206 is mounted within cover 201. Diaphragm 201 may move or deform in response to pressure incident on the diaphragm pressure sensing surface 208. An attachment mechanism as described above helps keep the sensor from moving in the body tissue and thus reduces spurious pressures on the diaphragm pressure sensing surface from movement of the sensor. Cover 201 extends past diaphragm 206 and may protect the diaphragm from impingement. Inlets 210 are shown positioned in the cover 201 at a location in front of diaphragm sensing surface 208. The inlets 210 may enhance fluid communication between the sensor and the surrounding bodily tissue.

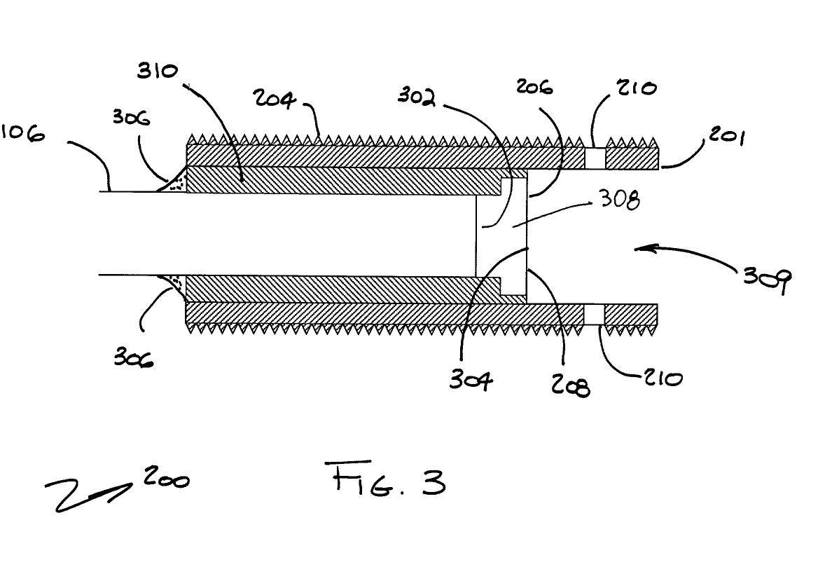

FIG. 3 illustrates a cross-sectional view taken along dotted line 3-3 of FIG. 2. The microsensor includes optical waveguide 106 having a polished or cleaved distal end 302. In this implementation, a housing 310 is attached to and surrounds the waveguide 106. A cover 201, which may be a capillary tube, surrounds the housing 310. The capillary tube may be made of quartz, stainless steel or other body implantable material. Mounted within the cover, and mounted to the housing, is diaphragm 206. The housing 310 may be attached to the cover 201 by any suitable means such as with an adhesive 306. The capillary tube cover 201 extends past the diaphragm 206 and serves to isolate and protect the diaphragm from impingement when installed in-vivo.

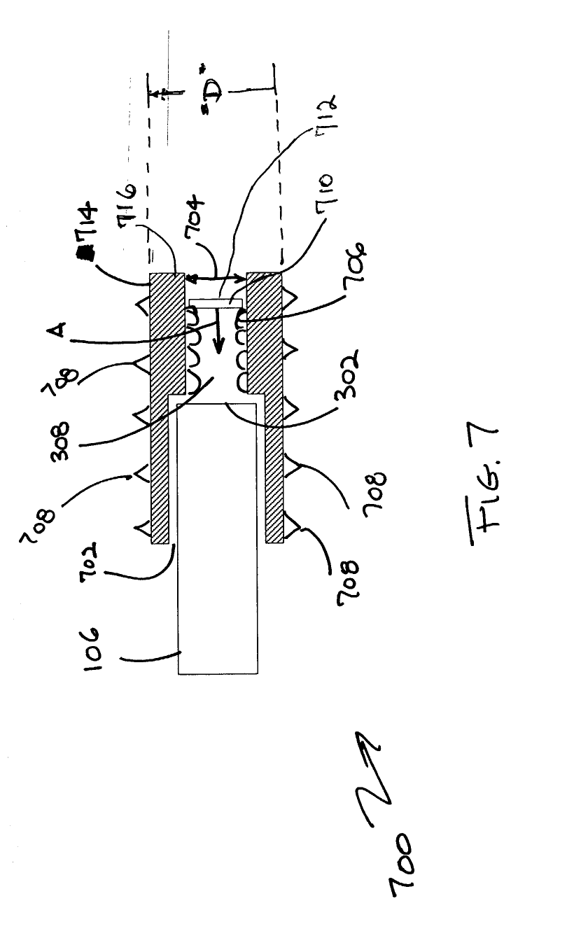

The outer surface of the capillary tube cover 201 may be altered to stabilize the microsensor in the bodily tissue. For example, the outer surface may be roughened or a wire may be attached as illustrated and described in FIG. 6. In the illustrated implementation, cover 201 is provided with a threaded surface 204 which may engage the bodily tissue when the sensor is implanted.

Diaphragm 206 is attached to the housing 310 by any suitable method, such as adhesive or anodic bonding. In an unstressed state, diaphragm 206 is displaced from, and parallel to, distal end 302 of the waveguide 106. Diaphragm 206 may be made of polymer, metal, silicon or crystalline materials and may have optical coatings on a reflective surface 304 to improve performance of reflecting light back into the waveguide that would otherwise be emitted from the waveguide. Coatings may also be chosen to provide a measurement of temperature through known optical techniques such as optical path length multiplexing. Examples of optical coatings include polyimide, glass, gold, silver, stainless steel, silicon, and silicon carbide.

An optical cavity 308 is formed between the diaphragm reflective surface 304 and the waveguide distal end 302. In response to physiological pressure, diaphragm 206 assumes a curvature due to the pressure on the diaphragm, and consequently the reflective surface 208 moves towards the waveguide distal end 302.

The sensor 200 may include mechanisms to permit fluid to flow between the region external to the sensor and the region defined by the space 309 between the diaphragm pressure sensing surface 208 and the end of the capillary cover 201. In this one implementation, fluid inlet holes 210 are provided in the capillary cover 201. The inlets may be arranged around the circumference of the cover to permit fluid communication between the exterior of the cover and the diaphragm sensing surface 208. Such fluid communication helps to achieve pressure equilibrium between the region between the diaphragm sensing surface 208 and the biological tissue.

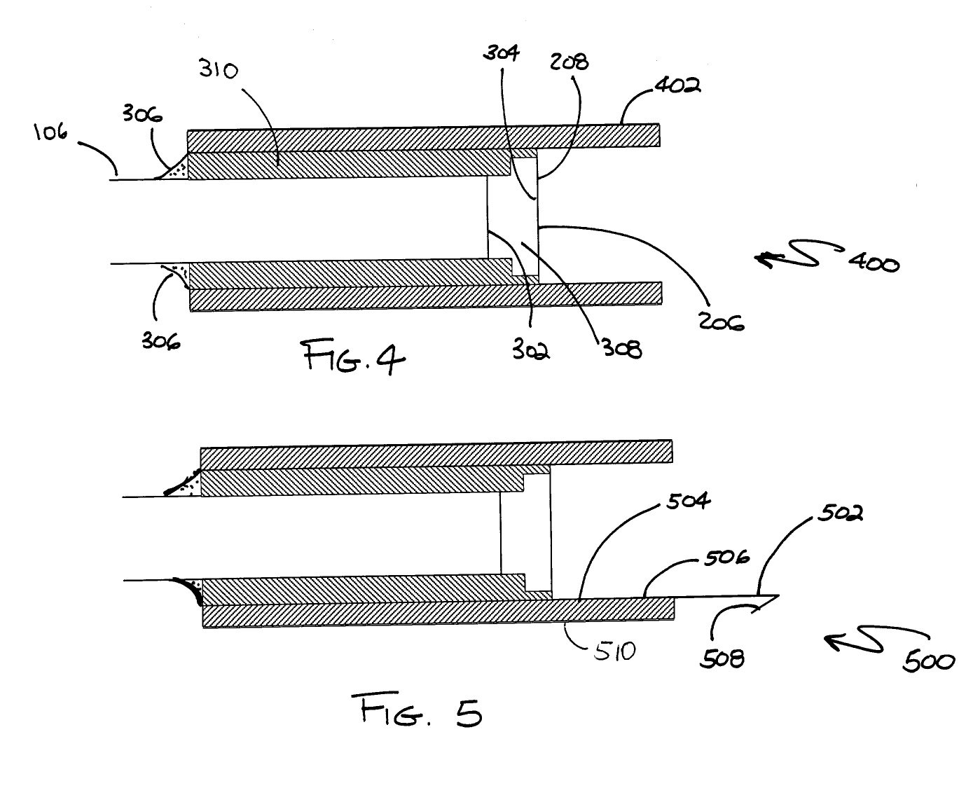

FIG. 4 illustrates an implementation of a sensor 400 having a waveguide 106 bonded by adhesive 306 to a housing 310. A diaphragm is attached to the housing as described above. A cover 402 is attached to the housing 310 and extends outwardly beyond the diaphragm 206, as shown. The cover 402 serves to isolate and protect the diaphragm from mechanical impingement when installed in-vivo. In this implementation cover 402 does not have a threaded surface or inlets as in FIG. 3. Diaphragm 206 has a reflective surface 304 displaced from, and parallel to, the polished distal end 302 of waveguide 106, and there is an optical cavity 308 between the reflective surface 304 and the distal end 302.

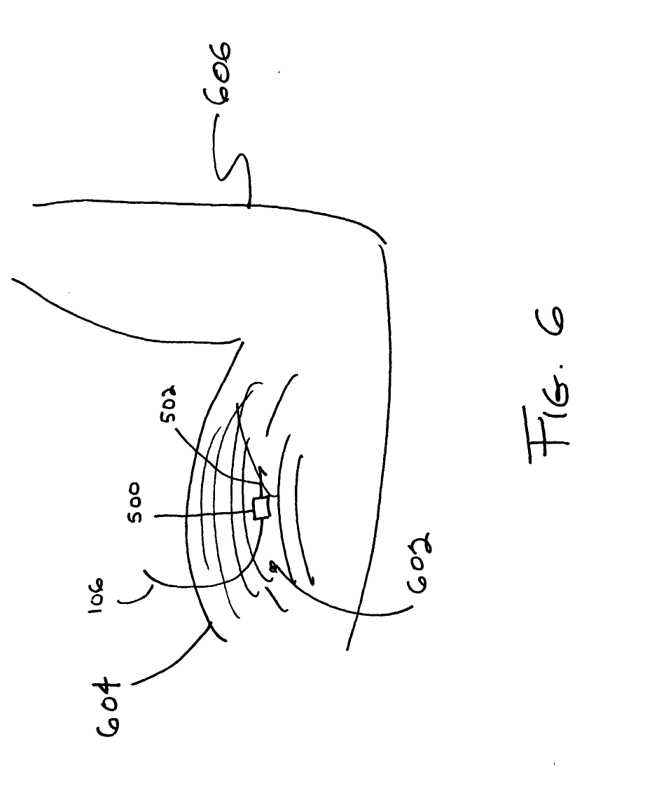

FIG. 5 illustrates another implementation of a sensor 500 similar to that of FIG. 4 but with the addition of an attachment mechanism 502 for stabilizing the sensor when implanted in bodily tissue. In this implementation, the attachment mechanism is a sensor stabilizing wire 502. One end 506 of wire 502 may be affixed to an inner wall of cover 510 by any suitable means, such as with an adhesive. Another end 508 of wire 502 is bent at a reflex angle to form a hook. In use, the wire extends from the sensor into the bodily tissue and anchors the sensor in a fixed position within the tissue. FIG. 6 illustrates the sensor 500 of FIG. 5 implanted in bodily tissue such as the bicep muscle 602 under skin 604 of a human arm 606. The microsensor may be inserted using a needle of appropriate dimensions. The needle is then withdrawn leaving the sensor embedded in the body. Other forms of implantation may be used that are known in the art.

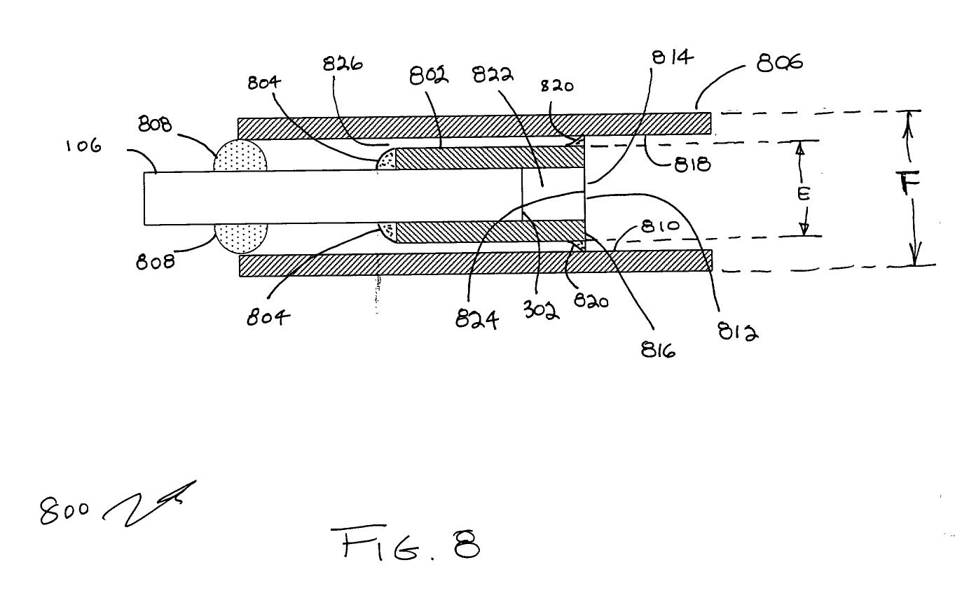

FIG. 7 illustrates an alternative implementation of a microsensor 700. In this embodiment, the diameter “D” of the microsensor is approximately 200 micrometers. Cover 714 has a first bore 702 that accepts an end of the optical waveguide 106. The waveguide is attached to the cover by any suitable means, such as with a bonding adhesive. Cover 714 may be made of polymer, metal, or crystalline materials with a coating on the exposed surfaces of the housing for improved adhesion with bodily tissue. Suitable cover materials may include, but are not limited to, polyimide, gold, silver, stainless steel, silicon, and silicon carbide. A second bore 704 in the cover creates a passage through the housing. Inside second bore 704, a first end of a bellows or other spring-like device 706 is attached near waveguide distal end 302 of the optical waveguide. A second end of the bellows is attached to diaphragm 710. The diaphragm 710 is not connected to the second bore 704 and is movable therein. The diaphragm 710 may be made, for example, of polymer, metal, or crystalline materials and may have optical coatings for enhanced performance. Suitable coating materials may include polyimide, glass, gold, silver, stainless steel, silicon, and silicon carbide. Bellows 706 and waveguide distal end 302 form an optical cavity 308. The bellows 706 may compress in response to pressures on the diaphragm pressure sensing side 712, thereby causing the diaphragm to move in the direction of arrow A. The bellows permits the diaphragm to be displaced relative to the optical waveguide in response to pressure while it maintains a parallel orientation to the distal end of the optical waveguide.

Cover 714 has a series of barbs or tines 708 on the outer surface of the cover to stabilize the microsensor in the bodily tissue. The barbs may extend around the entire circumference of the cover. Other stabilizing methods described herein may also be used.

The sensor 700 may include inlets (not shown) as illustrated in FIGS. 2 and 3 in the cover 714 to permit fluid to flow between the region external to the sensor and the region defined by the space between the diaphragm pressure sensing surface 712 and the end 716 of the capillary cover 714. Such inlets may be arranged around the circumference of the cover and permit fluid communication between the exterior of the housing the diaphragm sensing surface 712 to help achieve pressure equilibrium between the region between the diaphragm sensing surface 712 the bodily tissue.

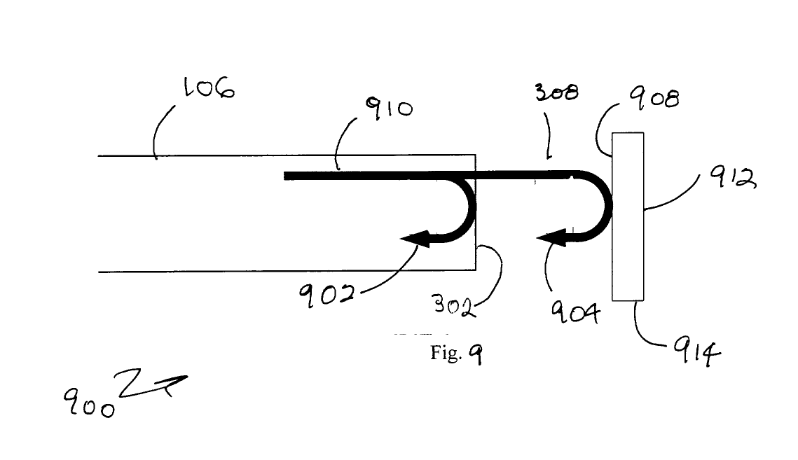

FIG. 8 illustrates an alternative implementation of a microsensor 800. The sensor comprises an optical waveguide 106 with a polished or cleaved distal end 302. Surrounding the waveguide 106 is a housing 802. Housing 802 is attached to waveguide 106 by any suitable means such as adhesive 804. Housing 802 extends beyond distal end 302 of waveguide 106. In a particular implementation, the housing may be a first capillary tube concentric with the waveguide. A cover 806 surrounds the housing 802 and is attached to the waveguide 106 by any suitable means such as adhesive 808. Cover 806 may extend beyond the waveguide distal end 302 and the housing 802. In a particular implementation, the cover may be a second capillary tube concentric with the first capillary tube and waveguide. Both the first and second capillary tubes may be made of quartz but this is not intended to be a limitation of the implementation. A diaphragm 812 is attached to a distal end 816 of the housing 802 and is attached to the interior wall of the cover 818 by any suitable method, such as by adhesive 820. The diaphragm has a pressure sensing surface 814 and a reflective surface 824 opposite to the pressure sensing surface. The diaphragm and waveguide distal end create an interference cavity 822. The diaphragm may be made of polymer, metal, or crystalline materials and may have optical coatings for enhanced performance. Specific material examples include polyimide, glass, gold, silver, stainless steel, silicon, and silicon carbide. As described herein, light emitted from the waveguide may be reflected off the reflective surface 824

The cover 806 serves to isolate and protect the sensor from mechanical impingement when installed in-vivo. A gap 826 may be provided between the cover 806 and the housing 802. Gap 826 may be filled or unfilled and serves to further protect the sensor from mechanical impingement by providing an area for dissipation or dampening of the impingement forces to prevent such forces from damaging the sensor. The outer surface of the cover 806 may be roughened, coated or provided with a profile as described above to stabilize the microsensor when implanted in the body. Additional stabilization of the sensor may be provided by a wire attached to the inner wall 810 of the cover 806 as described above. Inlets (not shown) as described above may also be formed in the cover 806 to permit fluid communication between the exterior of the sensor and the diaphragm sensing surface 814 and to help achieve pressure equilibrium between the region between the diaphragm sensing surface 814 and the ambient pressure.

In an unstressed state, diaphragm 812 is displaced from, and parallel to, the waveguide distal end 302. In response to physiological pressure, the diaphragm compresses toward the distal end of the fiber optic. Physiological pressure on the diaphragm pressure sensing surface 814 causes the diaphragm to assume a curvature toward the waveguide distal end 302 that alters the optical properties of the interference cavity 822. The altered optical properties change the interference pattern between the light emitted from the waveguide distal end 302 and the light reflected from the diaphragm reflective surface 824 as described in further detail below.

In this implementation the sensor diameter “E” without the cover 806 is approximately 360 micro-meters. The overall diameter “F” including the cover 806 is approximately 400 micro-meters.

FIG. 9 is a simple fiber optic arrangement 900 including a fiber optic waveguide 106 and a diaphragm 914 to help illustrate the concept of extrinsic Fabry-Perot interferometric (EFPI) fiber optic sensor technology. Operation of the EFPI is based on the relative displacement between a diaphragm reflective surface 908 and an optical waveguide polished or cleaved distal end 302. EFPI technology is a distance measurement technique based on the formation of a Fabry-Perot cavity 308 between the end face of a fiber optic waveguide 106 and a reflective surface 908. In operation, light 910 is passed through waveguide 106. A reference reflected portion 902 of the light reflects off the waveguide/medium, air for example, interface at waveguide distal end 302, while a main portion of the light propagates through the cavity 308 between the distal end and the reflective surface 908 where a sensing reflected portion 904 of the light is reflected back into the waveguide. These two light waves interfere constructively or destructively based on the path length difference traversed by the sensing reflection relative to the reference reflection, and travel back through the waveguide to a demodulation unit (not shown). The microsensor implementations described herein utilize diaphragm movements to measure pressure. Pressure in bodily tissue may be communicated to the diaphragm pressure sensing surface 912. The pressure causes movement of diaphragm 914 and alters the optical length of cavity 308. The change of the cavity length alters the interference relationship between the reference light waves and the sensing reflected light waves. The alteration of the interference may be related to the change in pressure.

Other implementations are within the scope of the following claims.

What is claimed is:

1. A body implantable sensor for sensing pressure in an environment within internal body tissue comprising:
a housing;
an optical waveguide attached to the housing;
a diaphragm having a reflective surface that faces a waveguide distal end and that reflects at least a portion of the signals exiting the waveguide distal end back into the waveguide, the diaphragm being movable relative to the waveguide distal end, wherein the distance between the waveguide distal end and the diaphragm reflective surface is determined by pressure forces acting on a sensing surface of the diaphragm that is opposite the reflective surface; and
a cover surrounding the diaphragm to protect the diaphragm from impingement, but leaving the diaphragm sensing surface exposed to pressure forces.

2. The sensor of claim 1 wherein a portion of the cover extends past the sensing surface of the diaphragm.

3. The sensor of claim 2 wherein the portion of the cover that extends from the waveguide distal end and past the diaphragm sensing surface includes inlets.

4. The sensor of claim 1 wherein a surface of the cover opposite the housing has at least one of threads formed thereon, tines formed thereon, barbs formed thereon and a coating.

5. The sensor of claim 1 further comprising a wire having a first end connected to the cover and a second hooked end.

6. The sensor of claim 1 wherein the diaphragm is approximately 11 micrometers thick and approximately 250 micro-meters in diameter.

7. The sensor of claim 1 wherein the sensor diameter is less than 400 micrometers.

8. The sensor of claim 1 wherein the diaphragm is approximately 2.4 micrometers thick and approximately 250 micro-meters in diameter.

9. A body implantable sensor for sensing pressure in an environment with internal body tissue comprising:
a pressure transducer; and
an attachment mechanism that is associated with the pressure transducer, wherein the attachment mechanism secures the sensor to the internal body tissue.

10. The sensor of claim 9 further comprising a cover surrounding the pressure transducer to protect the pressure transducer from impingement, but leaving the diaphragm sensing surface exposed to pressure forces.

11. The sensor of claim 10 wherein the attachment mechanism includes at least one of threads formed on the cover, tines formed on the cover, barbs formed on the cover and a coating on the cover.

12. The sensor of claim 9 wherein a portion of the cover extends past the sensing surface of the diaphragm.

13. The sensor of claim 12 wherein the portion of the cover that extends from the waveguide distal end and past the diaphragm sensing surface includes inlets.

14. The sensor of claim 9 wherein the attachment mechanism is a wire having a first end connected to the pressure transducer and a second hooked end.

15. A body implantable sensor for sensing pressure in an environment within internal body tissue comprising:
a housing;
an optical waveguide attached to the housing;
a diaphragm having a reflective surface that faces a waveguide distal end and that reflects at least a portion of the signals exiting the waveguide distal end back into the waveguide, the diaphragm being movable relative to the waveguide distal end, wherein the distance between the waveguide distal end and the diaphragm reflective surface is determined by pressure forces acting on a sensing surface of the diaphragm that is opposite the reflective surface; and
a cover surrounding the diaphragm to protect the diaphragm from impingement, but leaving the diaphragm sensing surface exposed to pressure forces, and having an attachment mechanism formed thereon that secures the sensor to the bodily tissue.

16. A body implantable sensor for sensing pressure in an environment with internal body tissue comprising:
a housing;
an optical waveguide attached to the housing;
a diaphragm having a reflective surface that faces a waveguide distal end and that reflects at least a portion of the signals exiting the waveguide distal end back into the waveguide, the diaphragm being movable relative to the waveguide distal end, wherein the distance between the waveguide distal end and the diaphragm reflective surface is determined by pressure forces acting on a sensing surface of the diaphragm that is opposite the reflective surface; and
a cover surrounding the diaphragm to protect the diaphragm from impingement, but leaving the diaphragm sensing surface exposed to pressure forces, and having a wire with a first end connected to the cover and a second hooked end the wire securing the sensor to the bodily tissue.

17. A body implantable sensor for sensing pressure in an environment within internal body tissue, comprising:
a housing;
an optical waveguide attached to the housing;
a deformable diaphragm having a reflective surface that faces a waveguide distal end and that reflects at least a portion of signals exiting the waveguide back into the waveguide, wherein the distance between the waveguide distal end and the diaphragm reflective surface is determined by pressure forces acting on a sensing surface of the diaphragm that is opposite the reflective surface; and
a cover surrounding the diaphragm to protect the diaphragm from impingement, but leaving the diaphragm sensing surface exposed to pressure forces.

18. A method of measuring physiological pressure comprising:
implanting an optical pressure sensor into an organism;
anchoring the sensor within the organism;
transmitting light into the sensor;
receiving a returned light signal; and
processing the returned light signal to determine the physiological pressure.

19. A method of measuring intramuscle pressure comprising:
implanting an optical pressure sensor into a muscle;
anchoring the sensor within the muscle;
transmitting light into the sensor;
receiving a returned light signal; and
processing the returned light signal to determine intramuscular pressure.

20. The method of claim 19 further comprising processing the intramuscular pressure to infer muscle force.

21. A method of measuring intramuscle pressure comprising:
fabricating an optical pressure sensor having a cover to protect the sensor from impingements;
implanting the sensor into an organism;
anchoring the sensor within the organism;
transmitting light into the sensor;
receiving a returned light signal; and
processing the returned light signal to determine the physiological pressure.

22. A system for measuring physiological pressure comprising:
a light source;
an optical coupler including a first waveguide coupled to the light source;
an optical sensor including,
a second optical waveguide;
a deformable diaphragm having a reflective surface that faces a distal end of the second waveguide and that reflects at least a portion of the signals exiting the second waveguide distal end back into the second waveguide, wherein the distance between the second waveguide distal end and the diaphragm reflective surface is determined by pressure forces acting on a sensing surface of the diaphragm that is opposite the reflective surface; and
a cover surrounding the diaphragm to protect the diaphragm from impingement, but leaving the diaphragm sensing surface exposed to pressure forces;
a spectrometer having a third waveguide coupled to the coupler; and
a signal processor coupled to the spectrometer.
